(19) United States
(12) Patent Application Publication
(10) Pub. No.: US 2021/0121428 A1
(43) Pub. Date: Apr. 29, 2021

(54) COMPOSITIONS FOR TREATMENT OF ANNULAR SPINAL DISC INJURY

(71) Applicant: Warsaw Orthopedic, Inc., Warsaw, IN (US)

(72) Inventors: Jared T. Wilsey, Memphis, TN (US); David S. Scher, Collierville, TN (US)

(21) Appl. No.: 16/664,164

(22) Filed: Oct. 25, 2019

(51) Int. Cl. A61K 31/198; A61K 9/00; A61K 31/401; A61K 47/08; A61K 47/12; A61K 47/26; A61K 47/36; A61K 35/28; A61K 38/27; A61L 27/54; A61L 27/58; A61L 27/38; A61L 27/20; A61M 5/178; A61M 5/142

(52) U.S. Cl. CPC A61K 31/198; A61M 2210/1003; A61K 31/401; A61K 47/08; A61K 47/12; A61K 47/26; A61K 47/36; A61K 35/28; A61K 38/27; A61L 27/54; A61L 27/58; A61L 27/3856; A61L 27/20; A61M 5/178; A61M 5/142; A61L 2430/38; A61L 2400/06; A61L 2300/802; A61L 2300/64; A61L 2300/604; A61L 2300/412; A61L 2300/252; A61K 9/0085

(57) ABSTRACT

This invention is directed to novel compositions containing a source of precursors to Type I collagen, an collagenic organic acid, an aldehyde, a polyol, an aldose, esters or salts or any combinations thereof, a blend of collagenic monomers or short-chain polymers, and a thickening agent to optimize the viscosity of the delivery system and methods of using such compositions for promoting collagen synthesis in the annular region of intervertebral disc.

### FIELD OF THE INVENTION

The present invention relates to biocompatible collagenic compositions containing material that enhances collagen synthesis for treatment of spinal disc defects.

### BACKGROUND

Spinal disc annulus is the tough, fibrous outer layer of an intervertebral disc. The annulus may be damaged from trauma, such as in acute disc herniation, or cumulative degenerative changes leading to significant (and symptomatic) annular fissures. Disc prostheses are devices that are implanted into the spine surgically when a spinal disc becomes damaged due to trauma or disease. These prostheses imitate the function of normal disc and preferably mimic the natural disc in shape and function. The drawbacks for such invasive procedure include but are not limited to potential infections, lengthy recovery, implant failure or fracture, narrowing of the spine or poor positioning of the implant leading to additional complications and continuous pain. As such there is a need in the art to offer the patients a treatment option that is less invasive and also improves patient outcome and recovery time.

### SUMMARY OF THE INVENTION

The present invention addresses the short comings of the prior art. The present invention is directed to novel methods for repairing annular damage by administering a novel delivery system that fosters collagen-synthesis at the defective region of an intervertebral disc. In at least one aspect, the inventive delivery systems of the present invention increases the access to collagen promoting molecules facilitating and enhancing the local collagen synthesis at the area of annular disc injury. The present methods, either singularly or in combination with other therapeutic agents or spinal sealants, adhesives or glues, permits the delivery of collagenic material to the damaged regions of the intervertebral disc thereby stimulating bone and tissue growth to repair and reverse the disc damage while prolonging the availability of molecules essential for synthesis of collagen in the area of interest.

In at least one aspect, the present invention is directed to injectable compositions for promoting collagen synthesis at the site of spinal injury containing a source of precursors for Type I, Type II, Type IV collagen and elastin fibers; a collagenic organic acid, an aldehyde, a polyol, an aldose, esters or salts thereof or any combinations thereof; a blend of collagenic monomers or short-chain polymers to preferably create a hypertonic environment; and a thickening agent to optimize the viscosity of the delivery system. In some embodiments, the present delivery systems allow small collagenic materials to be available to the local chondrocytes at the site of injury for a long enough time to initiate repair and reverse the annular disc damage.

In some embodiments, the source of precursors may be for synthesizing Type I collagen and is selected from the group consisting of gelatin, hydrolyzed collagen, glycine, proline, hydroxyproline, alanine, glutamate, tyrosine, aspartic acid, lysine, hydroxylysine, leucine, arginine, valine, threonine, phenylalanine, serine, histidine, tryptophan, methionine, cysteine, taurine, carnitine and any combinations thereof. In some embodiments, the sole amino acid precursors to in the delivery system is glycine, proline, hydroxyproline or a combination thereof.

In some embodiments, the organic acid is any one of lactic acid, pyruvic acid, citric acid, hyaluronic acid, oxalic acid, and malic acid, esters or salts thereof. In other embodiments, the organic acid is lactic acid, pyruvic acid or hyaluronic acid are the sole source of the organic acid source in the delivery systems of the present invention. In other embodiments, the delivery system of the present invention contains an aldehyde including but not limited to acetaldehyde, propionaldehyde, butyraldehyde and vanillin. In at least one embodiment, the sole source of aldehyde is acetaldehyde.

In some embodiments, the system may contain polyol such as glycerol, polyethylene glycol, xylitol, sorbitol, and mannitol. In some embodiments, the present system may contain an aldose. An aldose is simple sugar with a carbon backbone chain with a carbonyl group on the endmost carbon atom, making it an aldehyde. In some embodiments, the aldose used in the composition may be glyceraldehyde, erythrose, ribose, glucose, glactose mannose or allose.

In some embodiments, the thickening agent can be a polysaccharide, a protein, a starch, a vegetable gum, a sugar polymer, a glycosamineglycan and polyvinylpyrrolidone. In one embodiment, the polysaccharides is a sugar, a pectin, or a starch.

In some embodiment, the present system may further contain or be used in combination with a therapeutic agent, a contrast agent, a sealant, adhesive, glue or an osteogenic biologic. In some embodiments, the composition further contains a controlled release moiety to prolong the availability of any therapeutic agent to the local tissues.

In another embodiment, the delivery system may be a combination of a collagenic organic acid, an aldehyde, a polyol, an aldose, esters or salts thereof or any combinations thereof; a blend of collagenic monomers or short-chain polymers mixed with a hemostat, sealant or adhesive glue. In some embodiments, the system of the present invention includes a fibrin sealant having at least a two component material consisting of fibrinogen and thrombin that in the presence of small amounts of calcium and factor XIII, the thrombin converts fibrinogen into insoluble fibrin. In such embodiment, the delivery system contains fibrinogen, thrombin, an amino acid blend, an organic acid, aldehyde or polyol moiety, suitable collogenic monomers or short-chain polymers and a thickening agent. In some embodiments, the composition of the present invention is a freeze dried concentrate that may be reconstituted with a containing calcium salt solution.

In some embodiment the delivery system is a sterile composition containing polymeric cross-linked lyophilized fibrin network having lyophilized cross-linked fibrin polymer, lyophilized thrombin, a depot of an organic acid, an aldehyde, a polyol, or aldose and a blend of at least three (3) of any one of the material selected from the group consisting of gelatin, hydrolyzed collagen, glycine, proline, hydroxyproline, alanine, glutamate, tyrosine, aspartic acid, lysine, hydroxylysine, leucine, -arginine, valine, and threonine.

In some embodiments the composition may further contain hyaluronic acid, and a thickening agent. In some embodiments, wherein the organic acid, aldehyde, polyol or aldose is selected from the group consisting of pyruvate, lactate, acetaldehyde, polylactic acid, glycerol, and glucose. In some embodiments, the composition contains an amino acid that is glycine, proline, hydroxyproline, alanine or glutamate.

In one aspect, the invention is directed to a method for treating of a damaged intervertebral disc in a patient by delivering to region of the disc via a spinal cannula a composition containing a source of precursor to Type I collagen, an collagenic organic acid, an aldehyde, a polyol, esters or salts or any combinations thereof, a blend of collagenic monomers or short-chain polymers, and a thickening agent to optimize the viscosity of the composition.

In another embodiment, the present invention is directed to a method of treating a spinal injury or musculoskeletal tissue defect in a subject in need thereof by administering to the defective region of the tissue a composition having a source of precursor to Type I collagen, an ester or salt of an organic acid, an aldehyde, a polyol, an aldose or any combinations thereof, a blend of monomers or short-chain polymers, and a thickening agent. On other embodiments, the musculoskeletal tissue is an intervertebral disc tissue, cartilage, bone, muscle, or tendon, preferably an intervertebral disc. In some embodiments, the administration is by injecting the composition into the annular space of the intervertebral disc.

In another aspect, the present invention is directed to a kit comprising the composition containing a source of precursor to Type I collagen, a collagenic organic acid, an aldehyde, a polyol, an aldose, esters or salts or any combinations thereof; a blend of collagenic monomers or short-chain polymers; and a thickening agent to optimize the viscosity of the delivery system. In some embodiments, the source of precursors for Type I collagen is any one of gelatin, hydrolyzed collagen, glycine, proline, hydroxyproline, alanine, glutamate, tyrosine, aspartic acid, lysine, hydroxylysine, leucine, arginine, valine, threonine, phenylalanine, serine, histidine, tryptophan, methionine, cysteine, taurine, carnitine or any combinations thereof. In some embodiments, the kit includes a device for administering the composition to an annular defect of intervertebral disc and a pressure assisted pump to facilitate passage of the composition through the cannula. In other embodiments, the kit may further contain a therapeutic and/or an osteogenic agent.

In other embodiments, the present invention is directed to a kit comprising a fibrin sealant, a source of precursor to Type I collagen, a collagenic organic acid, an aldehyde, a polyol, an aldose, esters or salts or any combinations thereof, a blend of collagenic monomers or short-chain polymers, and a thickening agent with a device for administering the combination directly into the disc region having a tubing or cannula adapted to administer the composition into annulus region of the spinal disc.

### BRIEF DESCRIPTION OF THE DRAWINGS

The advantages of the invention will become apparent from the following detailed description of the preferred embodiments thereof in connection with the accompanying drawings, in which:

FIG. 1 illustrates an embodiment of the methods of the present invention and represents the image of annular delivery of the compositions of the present invention to an annular defect (left posterolateral) through a syringe system employed providing a non-surgical and minimally invasive application of the recently described delivery systems to an annular defect or the area at risk of damage.

### DETAILED DESCRIPTION OF THE INVENTION

This invention is directed to novel compositions and methods for promoting collagen synthesis using a composition containing a source of precursors to Type I, II, IV collagen or Elastin, a collagenic organic acid, an aldehyde, a polyol, an aldose, their esters or salts or any combinations thereof, a blend of collagenic monomers or short-chain polymers, and a thickening agent to optimize the viscosity of the delivery system.

As used herein, the term “delivery system” “composition,” or “formulation” may be used interchangeably to reflect the mixture material described in the present invention.

The term “treating” refers to a process wherein the delivery system of the present invention is injected into the damaged area or tissue to fill, seal, or plug the damaged area, enhance collagen synthesis in the region as compared to other sealants not containing the composition of the present invention, and/or reverse the progression of the damaged region by enhancing the local ability to initiate and maintain collagen and bone repair. Such process includes administering the delivery systems of the present invention to a subject (human or otherwise), in an effort to alleviate signs or symptoms of the disease. Alleviation can occur prior to signs or symptoms of the disease appearing, as well as after their appearance. Thus, “treating” or “treatment” includes “prophylactic treatment” of subjects at risk of developing spinal disc damage or injury.

The term “biocompatible polymer” refers to polymers which, in the amounts employed, are non-toxic and substantially non-immunogenic when used internally in the patient and which are substantially insoluble in the body fluid of the mammal. The biocompatible polymer can be either biodegradable or, a combination of biodegradable and low amounts of non-biodegradable biocompatible polymers.

The term “biocompatible solvent” refers to an organic material liquid at least at body temperature of the mammal in which the biocompatible polymer is soluble and, in the amounts used, is substantially non-toxic. Suitable biocompatible solvents may include, by way of example, water, ethanol, acetone, ethyl lactate, dimethylsulfoxide (DMSO), ethylene glycol, propylene glycol, glycerin, polyethylene or polypropylene glycols or analogues/homologues of anyone of such solvents. Aqueous mixtures with the biocompatible solvent may be a part of the optimal solvent mixture but in amounts that allows formation of the plug or the sealing of the damaged region. At least one example of biocompatible solvent include ethanol, dimethylsulfoxide, propylene glycol or mixtures thereof.

The term “collagenic” refers to material that can enhance synthesis and formation of Type I, Type II, and Type IV collagen. They include absorbable synthetic or natural material, polymers, agents and biologics.

The term “contrast agent” refers to a biocompatible radiopaque material capable of being monitored during injection into a mammalian subject by, for example, radiography, real time fluoroscopy, and the like. The contrast agent can be either water soluble or water insoluble.

The term “precursors” refers to material that promotes or supplements the synthesis of Type I, II and IV collagen.

The term “osteogenic biologics” refers to any material that can enhance collagen and bone formation, such as resorbable culture medium, tissue growth or differentiation factors including recombinant generated morphogenetic proteins, BMP, PDGF, TGF β, EGF/TGF-α, F-I, β FGF, hydrogels, absorbable natural polymers. Material such as lactic acid, glycolic acid or depots thereof, polylactic acid, polyglycolic acid, polytetrafluoroethylene, may also exhibit osteogenic properties.

The term “short-chain polymers” refers to amino acid polymers made up of repeating units containing 2 to 50 amino acids, preferably between 3 to 25 amino acids residues.

The term “Type I collagen” refers to the most abundant collagen and is the key structural composition of several tissues. It is expressed in almost all connective tissues and the predominant component of the interstitial membrane.

These desired ingredient materials may be adjusted as disclosed therein by one of skill in the art to create a delivery system suitable for promoting or prolonging synthesis of Type I, II, IV collagen or Elastin in the annular region of the intervertebral disc. One of skill in the art knows how to make alternative compositions which are also suitable for use in the present invention. The primary method for increasing the availability of the desired ingredients at the region of interest is to provide compositions that promote collagen synthesis.

In at least one aspect of the present invention, the delivery system of the present invention is an injectable composition containing a source of precursors for Type I, II and IV collagen, a collagenic organic acid, an aldehyde, a polyol, esters or salts thereof or any combinations thereof, a blend of collagenic monomers or short-chain polymers and a thickening agent to optimize the viscosity of the delivery system. In some embodiments, the source of precursors for Type I collagen is any one of gelatin, hydrolyzed collagen, glycine, proline, hydroxyproline, alanine, glutamate, tyrosine, aspartic acid, lysine, hydroxylysine, leucine, arginine, valine, threonine, phenylalanine, serine, histidine, tryptophan, methionine, cysteine, taurine, carnitine or any combinations thereof. In some embodiments, the amino acid precursor to Type I collagen is glycine, proline or hydroxyproline as the sole amino acid precursor source. In some embodiments, the source of precursors are for Elastin.

In certain embodiments, the organic or aldehyde source may be a lactate, a product of anaerobic metabolism, as well as acetaldehyde, pyruvate, and glycerol. In certain embodiments, the collogenic organic acid can be in the form of a polymer or organic acid free ester depot, such as polyglycolic acid, or polylactic acid or combinations thereof, whereby such depot or polymers may be hydrolyzed to their respective acid or ester moieties within the area of interest.

In some embodiments, a simple sugar (e.g., glucose) will be added to the delivery system to achieve a tacky, glue like consistency to promote retention in the annular defect. A blend of monomers and short-chain polymers of the collagenic metabolite(s) may contribute to a depot effect while also adding to the tackiness of the biologically active “glue.” Gelatin, hydrolyzed collagen, is also a rich source of the amino acid components of collagen and may be added to achieve an ideal viscosity for retention around the margins of the annular tear.

In some embodiments, the viscosity of such the delivery system may be increased by increasing the weight percent of the collagenic material in the composition; although, one of skill in the art also knows that modifying other parameters such as increasing the average molecular weight of the biocompatible polymer will increase viscosity. According to the present invention, a sufficient amount of the composition is introduced into the damaged region of the spine via a catheter delivery means until the desired volume is delivered to the region. In one embodiment, the administration is done under a fluoroscopy procedure.

In certain embodiment, the injectable delivery systems of the present invention contains (a) a source of precursor to Type I collagen that is selected from the group consisting of gelatin, hydrolyzed collagen, glycine, proline, hydroxyproline, alanine, glutamate, tyrosine, aspartic acid, lysine, hydroxylysine, leucine, arginine, valine, threonine, phenylalanine, serine, histidine, tryptophan, methionine, cysteine, taurine, carnitine and any combinations thereof; (b) an organic acid source such as lactic acid, pyruvic acid or hyaluronic acid, an aldehyde source that is selected from the group consisting of acetaldehyde, propionaldehyde, butyraldehyde and vanillin, an aldehyde source that is selected from the group consisting of glycerol, polyethylene glycol, xylitol, sorbitol, and mannitol or an aldose source such as glyceraldehyde. In some embodiments, the injectable delivery system is a composition that also contains a thickening agent that is selected from the group consisting of polysaccharides, proteins, starches, vegetable gums, sugar polymers, glycosamineglycans and polyvinylpyrrolidone. In some embodiments, the polysaccharide or sugar polymers are any one of chitosan, agar, carboxymethyl cellulose, pectin, carrageenan, aggrecan and versican or any combinations thereof.

In certain embodiment, the delivery system is a homogenous gel within the temperature ranging from about 20 to about 30° C. In certain embodiments, the source of precursor to collagen is in amounts ranging from 0.1 to about 45%, preferably between 1 to about 35% weight. In some embodiments, the source of precursor may be present in amounts of about 5%, 10%, 15%, 20%, 25% and 30% by weight. In certain embodiments, the organic acid, aldehyde, polyol and/or aldose individually or in combination may be present in amount ranging from 0.5 to about 45% weight. In some embodiments, the organic acid, aldehyde, polyol and/or aldose individually or in combination may be present in amounts of about 1%, 5%, 10%, 15%, 20%, 25%, 30%, 35%, and 40% weight. In certain embodiment, the thickening agent is present in an amount ranging from 0.1 to about 10% weight. In certain embodiment, the pH of the composition ranges from 5.5 to about 7.5.

In some embodiments, the composition contains a blend of glycine, proline and/or hydroxyproline in total amounts ranging from 0.5 to about 40% weight, preferably in amounts ranging from 1 to about 35% weight. In certain embodiments, the composition contains acetaldehyde, lactic acid, pyruvic acid, citric acid, and/or hyaluronic acid, glycerol, an aldose, salts or esters thereof alone or in combination in amounts ranging from 0.5 to about 35% weight or preferably in amount ranging from 2.5 to about 25% weight. In some embodiments, the composition contains acetaldehyde, lactic acid, pyruvic acid, citric acid, and/or hyaluronic acid, glycerol, an aldose in amounts of about 1%, 5%, 10%, 15%, or 20% weight. In certain embodiments, the thickening agent may include starches, vegetable gums, sugar polymers, glycosamineglycans and/or polyvinylpyrrolidone or any combinations thereof in amounts ranging from 0.1 to about 10% weight or preferably about 0.5 to about 7.5% weight. In some embodiments, the polysaccharides is chitosan, starch, pectin, or glucose.

In alternative embodiments, the delivery system may be contain or be combined with a therapeutic agent or an osteogenic biologic. The therapeutic agent of choice include an antibacterial agent, an antiviral agent, an anti-inflammatory agent, an antineoplastic agent, an oxysterol, a statin and an immunosuppressant. The osteogenic biologic may include resorbable culture medium of cells, disc chondrocytes, tissue growth or differentiation factors including recombinant generated morphogenetic proteins, BMP, PDGF, TGF β, EGF/TGF-α, F-I, β FGF. Other osteogenic material may include hydrogels, absorbable synthetic or natural polymers. Material such as lactic acid, glycolic acid or depots thereof, polylactic acid, polyglycolic acid, polytetrafluoroethylene, may also exhibit osteogenic properties. In certain embodiment the delivery system may contain a combination of a therapeutic agent and an osteogenic biologic. In one embodiment, the therapeutic agent may be a statin and the osteogenic biologic may be a fibrin sealant or a combination of fibrin and thrombin. In certain embodiments, the delivery system may be in combination with a scaffold or may be dispersed uniformly in a scaffold component.

In certain embodiments, the culture medium of cells may be from a tissue selected from the group consisting of bone marrow, adipose, muscle, brain, skin, liver, vascular smooth muscle, endothelium, blood, or placenta. In some embodiments, the cells could also be primary cells, differentiated cells, genetically modified cells, hybridomas, immortalized cells, transformed cells, tissue fragment cells, organelles, stem cells or a mixture thereof, nucleated cells, enucleated cells, germ cells, platelet cells, matrix vesicles, cell vesicles, demineralized bone paste, bone chips, cartilage fragments, or cell fragments or tissue fragments, as well as autologous cells, allogeneic cells.

In some embodiments, the composition may contain a platelet plasma component which may or may not be in the form of a lysate, to facilitate for example the release/provide growth factors and cytokines for tissue regeneration at the damaged area in need of repair. The present composition can further reduce inflammation or attract/mobilize cell signaling. In at least one embodiment, the present composition can initiate fibroblast repair of damaged annulus through fibroblast growth factors (FGF) or stabilize disc annulus and repair an annulus disc tears. In other embodiments, the present composition can stimulate revascularization to a disc; and/or stimulate stem cell activation. In other embodiments.

A simple sugar (e.g., glucose) will be added to the cocktail to achieve a tacky, glue like consistency to promote retention in the annular defect. A blend of monomers and short-chain polymers of the collagenic metabolite(s) may contribute to a depot effect while also adding to the tackiness of the biologically active glue. The short-chain polymers preferably is a 2 to 50 chain. Any of the 20 amino acids may be present, although charged amino acids would usually be present in fewer than 30%, frequently in fewer than 15%, by number in a repetitive sequence. In some embodiments, the short chain amino acid may have a unit containing at least three (3) amino acid residues selected from the group consisting of proline, glycine, hydroxyproline, alanine, glutamate, tyrosine, lysine, hydroxylysine and serine. These short chain polymers may be characterized by having a molecular weight ranging from about 5 to about 100 kD (kiloDaltons), more usually in the range of about 7 to about 40 kD and preferably about 15 to about 25 kD. The short chain polymers may be a tripeptide triad sequences found in natural collagens, particularly mammalian collagen.

In certain embodiments the delivery system may also contain hyaluronic acid (HA)-gelatin-containing poloxamer hydrogels. In such scenario, the compositions may contain a cell culture, allowing cell and/or tissue growth in three-dimensional matrices of varying stiffness. In certain embodiment the compositions of the present invention may include poloxamer, hyaluronic acid, gelatin, fibronectin, a peptide fragment of fibronectin, or any combination thereof. Suitable poloxamer includes polyoxypropylene (having a molar mass of about 1,000 to about 3,500 g/mol) and polyoxyethylene, and the composition can transition from a liquid at cooler temperatures (e.g., about 4° C.) to a gel at warmer temperatures (e.g., about 37° C.).

In certain embodiments, the composition may contain a controlled release moiety to prolong the availability of any therapeutic agent to the local tissues. Controlled release compositions of the present invention can include a polymeric matrix forming material including but not limited to poly(lactide-co-glycolide) or poly lactic acid polymers and copolymers having molecular weight of less than 100,000 kD, mineral oil, sesame oil, beeswax, propyl glycol, olive oil, soybean oil, oil, wheat germ oil grapeseed oil, sunflower oil, castor oil, linseed oil, soybean oil, corn oil, coconut oil, palm oil, oil walnut, hazelnut oil, rapeseed oil or squalene or olive squalene. The collogenic material may be released from any such matrix by diffusion or by biodegradation of the matrix material or both.

Delivery of the compositions of the present invention is via a suitable delivery device to the site of injury such as a spinal cannula, medical catheter, applicator or a spine needle. The device employed is not critical provided that device components are compatible with the composition (i.e., the device does not degrade the composition) and of sufficient strength. Materials compatible with the compositions can be readily determined by the skilled artisan and include, for example, polyethylene, other polyolefins, fluoropolymers (e.g., Teflon.™), silicone, stainless steel or alike. In certain embodiments, the delivery device may be heated or warmed to allow delivery of the composition to the site of interest. It is important in practicing the methods of the present invention that the injection syringe and other portions of the apparatus have sufficient strength to withstand the pressures required to flow a viscous composition through the catheter. The pressure may exceed 1000 pounds per square inch (psi) when using a composition with a viscosity of about 2500 centistokes.

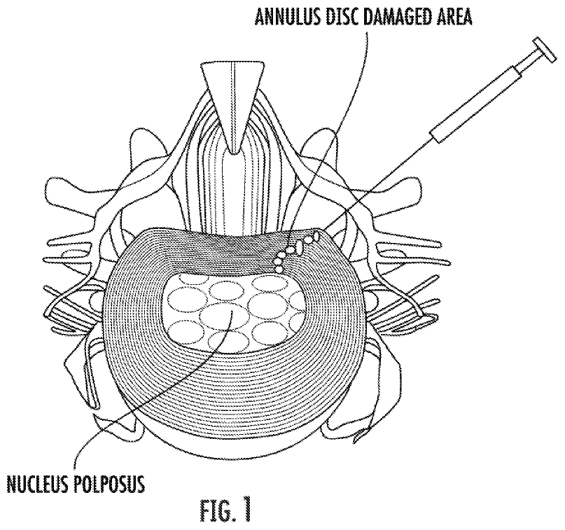

In some embodiments, the cannula may be adapted to engage with annulus annular space of the intervertebral disc having suitable dimension for delivery of such composition to the desired space. In some embroilments the preferred delivery device may be a cannula, needle or applicator having a length ranging from 5 mm to about 750 mm, a diameter of ranging between 0.1 mm to about 20 mm having able to deliver a volume ranging from 0.05 ml to about 10 ml. In some preferred embodiments, the cannula, needle or applicator has a length ranging from 50 mm to about 400 mm, a diameter of ranging between 1 mm to about 7 mm having able to deliver a volume ranging from 0.05 ml to about 5 ml. In certain embodiments, the delivery systems of the present invention are injected into a damaged disc in a percutaneous procedure. See FIG. 1. One of skill in the art will recognize alternative devices and components that are suitable for this purpose.

Suitable biocompatible solvents for delivery of the blend in the composition of the present invention in include water, ethanol, acetone, ethyl lactate, dimethylsulfoxide (DMSO), polyethylene glycol, polypropylene glycol, glycerin, analogues/homologues of dimethylsulfoxide or any mixtures thereof. Aqueous mixtures with the biocompatible solvent may be a part of the optimal solvent mixture but in amounts that allows formation of the plug or the sealing of the damaged region.

In certain embodiments, a uniform suspension of contrast agent and biocompatible polymer in the biocompatible solvent is desirable for use in the methods of the invention to allow visualization of the delivery during the procedure.

In a preferred embodiment, the invention relies on the ability of specific metabolites to promote collagen synthesis. These collagenic compounds will be combined in a blend that creates a sticky annular composition that serves to stabilize the annular fissure during the biological repair process while creating a depot of the active ingredients. In one embodiment, monomers and short polymers or depot compositions of pyruvate, lactate, and/or acetaldehyde at concentrations ranging between 1 to 25%, preferably 5%, 10%, 15%, and 20% weight and may further be included in a tacky blend with glucose and glycerol. Amino acid precursors of collagen synthesis (e.g., glycine, proline, hydroxyproline, alanine, and glutamate) may also be included at concentrations ranging between 1% to 10% weight to support the endogenous healing process.

In another aspect of the present invention, a method of treating a musculoskeletal tissue defect is described for that includes the steps of administering to the defective region of the tissue a composition comprising: a) a source of precursor to Type I collagen, b) an ester or salt of an organic acid, an aldehyde, a polyol or any combinations thereof, c) a blend of monomers or short-chain polymers, and d) a thickening agent. The damaged musculoskeletal tissue include an intervertebral disc tissue, cartilage, bone, muscle, or tendon. In one embodiment, the compositions of the present invention are administrated into the annular space of the intervertebral disc to prolong the availability of the collagenic material to the local tissue and cells to initiate repair.

In certain embodiments, the present invention provides the use of the composition of the present invention for soft tissue repair, for site-specific delivery of osteogenic biologic, for bone repair, for repairing or resurfacing damaged cartilage or for repairing meniscus that provides an enhanced collagen synthesis at the area of injury as compared to the fibrin sealant or other spinal adhesives that do not contain the instantly claimed blend of collagenic material. In one embodiment, the compositions of the present invention is administered directly into an annular defect of the intervertebral disc. In certain embodiments, the administration of the presently described compositions to the site of injury can be accomplished manually or via a pressure assisted pump. Proper dosages can be ascertained by one of skill in the art using the teachings of this disclosure and readily available literature.

As shown in FIG. 1, the spinal needle is inserted into the defect that is located at the posterior lateral location of the annulus of the disc. Standard procedures can be employed to position the distal (i.e., tip) of needle into the damaged area. The proximal end of the delivery needle maybe connected to one or more syringes or pumps to facilitate steady pressure for delivery of the composition to the area. In some embodiments, the delivery device may be hydrated or warmed to facilitate ease of delivery to the region of interest.

The present invention also describe methods of manufacturing the present delivery systems and kits containing the composition of the present invention, a suitable device for administering the composition to an annular defect of intervertebral disc and optionally a pressure assisted pump to facilitate passage of the composition through the cannula. Methods of manufacturing may include general steps known in the art to combine the osteogenic material described herein.

In an alternative embodiment of the invention the may further comprise the step of suturing or placing against the damaged area in the patient's annulus a platelet rich plasma combined with calcium and thrombin or fibrin glue generally known in the art to seal the annular tissues in the damaged region of the spine. Additionally, by combining platelet therapy with stem cells, there can be synergy with respect to reducing back pain.

While the invention has been described in detail with reference to preferred embodiments thereof, it will be recognized that the methods of the invention may be applied to the treatment of other sites in need of such treatment. Further, it will be apparent to one skilled in the art that various changes can be made, and equivalents employed, without departing from the scope of the invention.

1. An injectable composition for promoting collagen synthesis comprising:
a) a source of precursor to collagen or elastin,
b) an ester or salt of an organic acid, an aldehyde, a polyol or any combinations thereof,
c) a blend of monomers or short-chain polymers, and
d) a thickening agent.

2. The composition of claim 1, wherein the source of precursor is for Type I collagen and is selected from the group consisting of gelatin, hydrolyzed collagen, glycine, proline, hydroxyproline, alanine, glutamate, tyrosine, aspartic acid, lysine, hydroxylysine, leucine, -arginine, valine, threonine, phenylalanine, serine, histidine, tryptophan, methionine, cysteine, taurine, carnitine and any combinations thereof.

3. The composition of claim 2, wherein the precursor for Type I collagen is glycine, proline or hydroxyproline.

4. The composition of claim 1, wherein the organic acid is selected from the group consisting of lactic acid, pyruvic acid, citric acid, hyaluronic acid, oxalic acid, and malic acid.

5. The composition of claim 4, wherein the organic acid is lactic acid, pyruvic acid or hyaluronic acid.

6. The composition of claim 1, wherein the aldehyde is selected from the group consisting of acetaldehyde, propionaldehyde, butyraldehyde and vanillin.

7. The composition of claim 6, wherein the aldehyde is acetaldehyde.

8. The composition of claim 1, wherein the polyol is selected from the group consisting of glycerol, polyethylene glycol, xylitol, sorbitol, and mannitol.

9. The composition of claim 8, wherein the polyol is glycerol.

10. The composition of claim 1, wherein the thickening agent is selected from the group consisting of polysaccharides, proteins, starches, vegetable gums, sugar polymers, glycosamineglycans and polyvinylpyrrolidone.

11. The composition of claim 10, wherein the polysaccharides is starch, pectin, or glucose.

12. The composition of claim 10, wherein the sugar polymers are agar, carboxymethyl cellulose, pectin, carrageenan, aggrecan and versican.

13. The composition of claim 10, wherein the glycosaminoglycan comprises chondroitin-4-sulfate and chondroitin-6-sulfate.

14. The composition of claim 1, further comprising a therapeutic agent or an osteogenic biologic.

15. The composition of claim 14, wherein the therapeutic agent is selected from the group consisting of an antibacterial agent, an antiviral agent, an anti-inflammatory agent and an antineoplastic agent.

16. The composition of claim 14, wherein the osteogenic biologic is selected from the group consisting of stem cell and growth hormones.

17. The composition of claim 14, further comprising a polymer for controlling the release of the therapeutic agent or osteogenic biologic.

18. The composition of claim 1, further comprising a scaffold component.

19. A method of treating a musculoskeletal tissue defect in a subject in need thereof comprising administering to the defective region of the tissue a composition comprising: a) a source of precursor to Type I collagen, b) an ester or salt of an organic acid, an aldehyde, a polyol or any combinations thereof, c) a blend of monomers or short-chain polymers, and d) a thickening agent.

20. The method of claim 19, wherein the musculoskeletal tissue is an intervertebral disc tissue, cartilage, bone, muscle, or tendon.

21. The method of claim 20, wherein the musculoskeletal tissue is an intervertebral disc.

22. The method of claim 21, wherein the administration is by injecting the composition into the annular space of the intervertebral disc.

23. A method of treating an annular defect of an intervertebral disc in a subject in need thereof comprising
administering into the annular defect of the intervertebral disc a composition comprising a) a source of precursor to Type I collagen, b) an ester or salt of an organic acid, an aldehyde, a polyol or any combinations thereof, c) a blend of monomers or short-chain polymers, and d) a thickening agent and sealing the annular defect.

24. The method of claim 23, wherein the composition is administered through a cannula into the damaged disc space.

25. The method of claim 24, wherein the composition is administered into the posterolateral region of the intervertebral disc into the annular defect.

26. The method of claim 25, further comprising the step of prolonging the exposure of the ingredients to the defective region of the disc.

27. The method of claim 24, wherein the cannula is a spinal syringe.

28. A kit comprising the composition of claim 1 and a device for administering the composition to an annular defect of intervertebral disc and a pressure assisted pump to facilitate passage of the composition through the cannula.
